(19) United States
(12) Patent Application Publication
(10) Pub. No.: US 2025/0255602 A1
(43) Pub. Date: Aug. 14, 2025

(54) INSTRUMENT LARIAT PASSER FOR GUIDING AN INSTRUMENT TO A SURGICAL SITE, AND METHODS OF GUIDING AN INSTRUMENT TO A SURGICAL SITE

(71) Applicant: Amazing Brace LLC, Tampa, FL (US)

(72) Inventor: Victor Hayes, Tampa, FL (US)

(21) Appl. No.: 19/053,466

(22) Filed: Feb. 14, 2025

Related U.S. Application Data
(60) Provisional application No. 63/553,438, filed on Feb. 14, 2024.

(51) Int. Cl. A61B 17/04

(52) U.S. Cl. CPC A61B 17/0469

(57) ABSTRACT

Lariat passer devices include a guide, a first loop and a second loop, and a wire extending from one end of the guide to a handle at the other end of the guide; the handle includes a wire tightening or locking mechanism. Kits include the present devices and instructions for use of the device, a storage container, a cannula, a visualization tool, and/or an instrument to be delivered to a surgical site of a patient. Methods of delivering an instrument to a surgical site of a patient include inserting a cannula to a surgical site; adding a first loop of a lariat passer device to the cannula; adding a second loop of the device over an instrument to be delivered to the surgical site; tightening the first and second loops over the cannula and the instrument; and guiding the instrument along the cannula to the surgical site.

### FIELD

The present application relates to devices, systems, and methods of providing instruments to surgical sites, such as scopes and complementing instruments, for example for endoscopic surgery.

### BACKGROUND

In endoscopic surgery, such as spinal surgery, the placement of instruments in a patient is often limited by a need to insert the instrument to a surgical site using a cannula or portal around the instrument. There is a need for improved methods of guiding devices to surgical sites.

### BRIEF SUMMARY

Provided herein are devices, systems, kits, and methods for guiding instruments to a surgical site, for example, in minimally invasive surgeries. By way of non-limiting example, the present devices, systems, and methods may be used in endoscopic surgery, for example to extremities and spinal surgery on a patient. The present devices, systems and methods may be used in other types of surgery at other sites as well. Also provided are kits that include one or more of the present devices and optionally at least one additional component related to use of the device.

Example devices and systems include lariat passer devices including a guide having a body and a handle, a first loop and a second loop, wherein the first loop and the second loop are at a first distal end of the guide, and a wire extending from the first distal end of the guide to a second distal end of the guide. In example embodiments, the handle is at the second distal end of the guide; the handle includes a wire tightening and/or locking mechanism.

Example kits include at least one of the present lariat passer devices and at least one additional component such as instructions for use of the device, a storage container, a cannula, a visualization tool, and/or at least one instrument to be delivered to a surgical site of a patient.

Example methods include treating a patient by utilizing the present devices and systems to guide one or more instruments to a surgical site. According to non-limiting example embodiments, the present methods include methods of performing endoscopic surgery on a patient, for example on the spine or extremities of a patient, while utilizing the present devices and systems. Example methods of delivering an instrument to a surgical site in a patient include inserting a cannula to a surgical site in a patient; adding a first loop of a lariat passer device as provided herein, to the cannula; adding a second loop of the lariat passer device over a first instrument to be delivered to the surgical site; tightening the first loop over the cannula and tightening the second loop around the first instrument to be delivered; and guiding the first instrument to be delivered along the cannula to the surgical site of a patient. A third or more loop may be provided, to deliver a second or more instrument to the surgical site.

### BRIEF DESCRIPTION OF THE DRAWINGS

FIG. 1 depicts a non-limiting example of a device in accordance with embodiments of the present application.

FIG. 2 depicts a non-limiting example of a device in accordance with other embodiments of the present application.

FIG. 3 depicts another non-limiting example of a device in accordance with embodiments of the present application.

FIG. 4 depicts a further non-limiting example of a device in accordance with embodiments of present application.

FIG. 5 depicts an optional pop over handle with a ratcheting system for tightening a lariat of devices according to example embodiments of the present application.

FIGS. 6A and 6B depict how the present devices and systems may be used to guide an instrument to a surgical site in a patient through a small incision in minimally invasive surgery.

FIG. 7 depicts insertion of an endoscope with a standard medical tube or cannula, including a novel lariat passer in accordance with non-limiting embodiments of the present application.

FIG. 8 depicts a non-limiting embodiment of a device in accordance with the present application, which may be used to guide instruments to a surgical area in a patient.

FIG. 9 depicts another example embodiment of a device in accordance with the present application.

FIGS. 10A and 10B depict further example embodiments of a device in accordance with the present application, with and without an optional guide.

### DETAILED DESCRIPTION

The terms “a” or “an”, as used herein, are defined as one or more than one. The term plurality, as used herein, is defined as two or more than two. The term “another”, as used herein, is defined as at least a second or more. The terms “including” and “having,” as used herein, are defined as comprising (i.e., open language).

According to example embodiments, the present application includes devices, systems, kits, and methods that facilitate or allow access to a surgical site (including, but not limited to, surgical sites in extremities and/or the spine) of a patient with an instrument that is desired or needed to be at the surgical site of the patient, by guiding the instrument to the site. The instrument may be lassoed to a cannula or other device that has already been inserted into a patient, and the instrument is guided along the already inserted device to the surgical site.

The present devices, systems and methods allow minimally invasive sliding of instruments through skin and guide them to a desired location in the patient, such as a camera site (e.g., at the end of a cannula), without the use of an additional cannula or portal needing to be around the instrument. This allows free maneuvering/motion of working instruments without the restrictions of a canula.

The term “patient” as used herein is intended to include a mammal receiving medical treatment. The mammal may include for example a human, but is not limited thereto.

The term “surgery” is not limited to a particular type of surgery, and may include, but is not limited to, open or minimally invasive surgery, such as endoscopic surgery. The location of a surgery is not limited, and may be performed in the hospital, or it may be within a surgery center, doctor's office, or elsewhere.

The terms “lariat”, “loop”, and “lasso” are used interchangeably herein.

### Devices and Systems

Non-limiting examples of the present application include devices, systems and methods that facilitate movement and/or positioning of an instrument to a surgical site within a patient. The devices, systems and methods may be referred to herein somewhat interchangeably, for example, as an Instrument Passer, Instrument Lariat Passer, or Supplemental Access for Spine: Scopes and Instruments (SASSI), but these terms should not be considered to limit any particular embodiments. It should be understood that the name of the present devices and systems may change and still be within the scope of the present application.

Example devices and systems provided herein include lariat passer devices including a guide having a body and a handle, a first loop and a second loop, in which the first loop and the second loop are at a first distal end of the guide, and a wire extending from the first distal end of the guide to a second distal end of the guide. In example embodiments, the handle is at the second distal end of the guide. The handle may include a wire tightening and/or locking mechanism. In example embodiments, the present devices and systems may include a third or more loop, for example when they are configured to provide two or more instruments to a surgical site of a patient.

According to non-limiting example embodiments, the present devices include a first loop configured in size, shape and material to be suitable to loop over a device within a patient, such as a cannula having an end at or near a desired surgical site in a patient; and a second loop configured in material, size and shape to loop over an instrument to be delivered to a surgical site. According to non-limiting example embodiments, the second loop over the instrument to be delivered, may be a rough material, and/or be coated in a rough material such as silicon or other material that would prevent the instrument from sliding, or reduce sliding. According to example embodiments, the first loop over the cannula that already has an end at an appropriate surgical site inside tissue (such as a scope cannula) or that will be inserted to the surgical site first, comprises a smoother or slicker material than a material or coating of the second loop, which allows guiding or sliding of the guide and instrument to the surgical site. The present devices and systems may include a third (or more) loop, which may be used to deliver a further instrument to the surgical site. The third or more loop may be included and optionally attached to one or both of the first and second loops. The loops may be incorporated into an Instrument Lariat Passer device. The present devices may include a guide that has a narrowed end, such as a bullet-shaped end (or other suitable shape) to facilitate insertion of the instrument into skin, a handle, a tightening device for tightening the loops, and a locking mechanism.

According to example embodiments the first loop is looped over a cannula (e.g., a camera tube, which may include e.g. a camera and/or inflow fluid therein) that is already at an appropriate surgical site deep inside tissue or is to be inserted into a patient to a surgical site before an instrument to be delivered. In non-limiting examples, there is one loop (the first loop) over this cannula.

Non-limiting examples of a Lariat Guiding Instrument Passer (or Lariat Passer) of the present application includes a loop locking and tightening device or system, a guide that may be a tissue dilator device with a distal lariat and wire (or other suitable material) that forms a lariat around the two or more objects. The present devices may also include a handle.

The term “wire”, as used herein is used generally to cover wire and non-wire material. The “wire” and/or loops/lariats may be made of various materials, non-limiting examples of which may include wires, stainless steel wire, wire of other alloys, fiber type wire or FIBERWIRE®, such as braided suture material, other suture material, or other suitable materials that may be apparent to those skilled in the art in view of the present disclosure.

The selection of wire material and/or coating may be based e.g. on desired strength, size, and/or friction. A wire may be of a suitable diameter and strength to achieve the present purpose of being able to advance an instrument to a surgical site within a patient.

As would be apparent to those skilled in the art, devices and wires herein may be a suitable size, configuration, shape, strength and/or material for the intended purpose. For example, the device and/or wire may be smaller and/or narrower for embodiments configured for minimally invasive purposes, than for non-minimally invasive purposes.

### Methods

Also included are methods that include using the present devices and systems to move and/or position an instrument to a surgical site within a patient.

Example methods of the present application include methods of delivering an instrument to a surgical site in a patient, which include inserting a cannula to a surgical site of the patient, looping a first loop of a lariat passer device of the present application over the cannula, in which the lariat passer device also has at least a second loop; looping the second loop over an instrument to be delivered to the surgical site, tightening both the first and second loops around both the cannula and the instrument to be delivered, and guiding or pushing the Lariat Passer device and the instrument to be delivered through tissue by sliding the lariat passer device and instrument along the length of the cannula (with a loop around the cannula) to the surgical site.

According to non-limiting example embodiments, looping a first loop of a lariat passer device over the cannula may be performed prior to inserting the cannula to a surgical site of the patient.

According to non-limiting example embodiments, the cannula may include for example a camera and/or inflow fluid therein. The present methods may include a third (or more) loop, which may be used to deliver a further instrument to the surgical site. In these embodiments, methods may further include looping the third loop over a second instrument to be delivered to the surgical site; tightening the third loop over the second instrument to be delivered; and guiding the second instrument to be delivered along the cannula to the surgical site of a patient. According to non-limiting examples, the loops may be tightened different amounts. For example, a loop around the cannula may be tightened less (to enable sliding), than a loop around an instrument to be delivered.

In example embodiments, the first loop may be made of and/or coated with a smoother material than a material and/or coating of the second loop.

There may multiple ways of tightening the loops, according to the present devices, systems and methods.

In addition to being described herein, various embodiments are depicted in the figures, discussed herein.

FIG. 1 depicts a first non-limiting example of an Instrument Lariat Passer device 1 in accordance with embodiments of the present application. FIG. 1 shows a non-limiting example of a device 1 having multiple locking systems or apparatuses. In this example device, a tissue dilator device or a guide is shown that includes a handle 3 and a body 2, a wire locking and/or tightening apparatus 4, first and second distal lariats or loops 5 (distal to the handle, for example), and wire 6 that is configured to be capable of forming two or more lariats or loops 5 around two or more objects, or is attached to two or lariats or loops 5. The lariat may be considered to be a second locking system in example embodiments.

Although in FIG. 1, the lariat includes two loops 5, non-limiting examples of lariats may include two or more lariats or loops, such as two, three, four or more lariats or loops.

The depicted example may include lariats 5 that may be made of, for example, silicon or elastic polymer, or include, for example, a silicon or elastic polymer covering or coating around one or both lariats. In example embodiments, the material and/or coating of lariats 5 may be selected to prevent the instrument from sliding after locking. Other materials and/or coatings suitable for insertion into a patient, are also envisioned and may be used in accordance with example embodiments. According to example embodiments, the lariats 5 may be a portion of the wire 6 or they may be attached to the wire 6. The lariats 5 may be the same or different material than the wire 6 (or the rest of the wire 6—in a case in which the lariat is part of the wire), and/or the lariats 5 may have the same or different coating from the wire 6 (or the rest of the wire). The first and second (or more) lariats 5 may also be formed of different or the same materials and may have different coatings from one another. In non-limiting examples, one or both of the lariats, or the lariats and the wire, may be coated, only partially coated, or not coated. According to example embodiments, the loops or lariats may be connected to one another and/or they may be connected to the guide, but not necessarily to one another.

According to example embodiments, the present devices may include a wire locking or tightening apparatus, configured to lock wires 6 at a desired location. In the depicted example of FIG. 1, the wire locking and/or tightening apparatus 4 is a toggle spring loaded locking mechanism 7 configured to lock in the wires (or other material). Example toggle spring loaded locking mechanism may be plastic and/or metal materials. A wire locking apparatus according to this and other example embodiments may include a device that may be reversibly lockable and unlockable.

The wire locking apparatus may be positioned for example, at or on the handle 3 or as part of the handle 3 of the present device, although the specific position on the handle is not limited.

In this and other examples herein, there may be additional ropes, loops and/or locks. Lockable rings can be used to sync and tighten the lariats/loops around instruments to slide them to the surgical site in a patient.

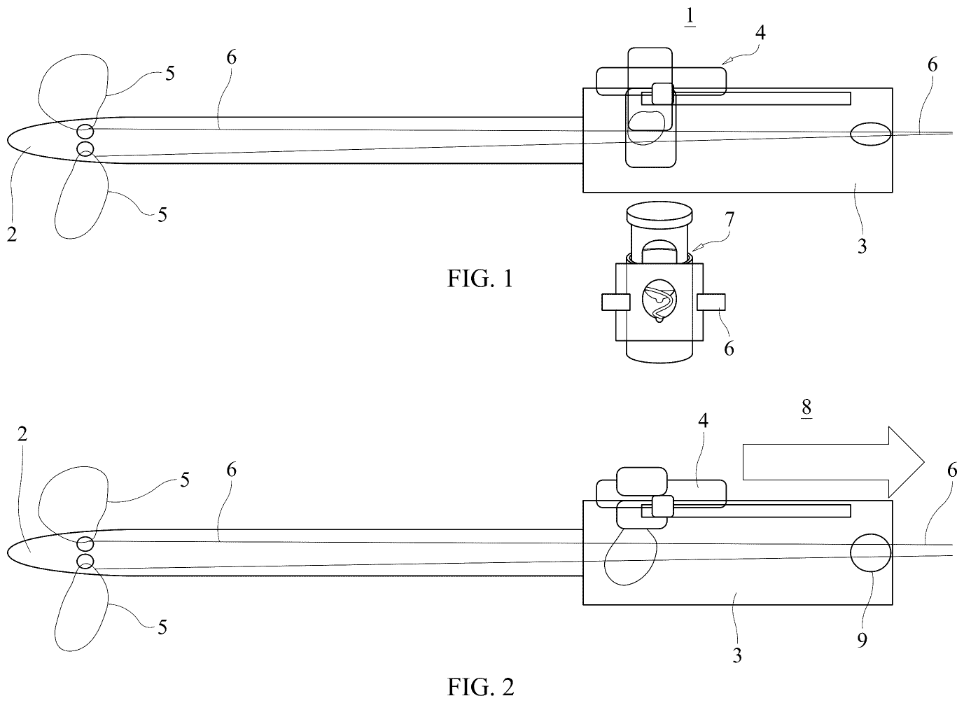

FIG. 2 depicts a second non-limiting example of an Instrument Lariat Passer device 8 in accordance with the present application. Unless otherwise indicated, the description of the same elements from FIG. 1 or other figures or embodiments herein, will not be repeated for this and additional embodiments herein. For example, the lariats and wires may be as described above.

FIG. 2 shows that in example embodiments, the handle may include a hole 9 where excess wire 6 (or other material) can be pushed into the device 8 to allow loops/lariats to enlarge and easily be passed over instruments.

FIG. 2 shows a wire locking apparatus 10 that may be a different type of wire locking apparatus than that show in FIG. 1. In example embodiments, wire locking apparatus 10 may be an apparatus configured to lock wires 6 into place, for example, by sliding a portion of the wire locking apparatus 10 along the handle.

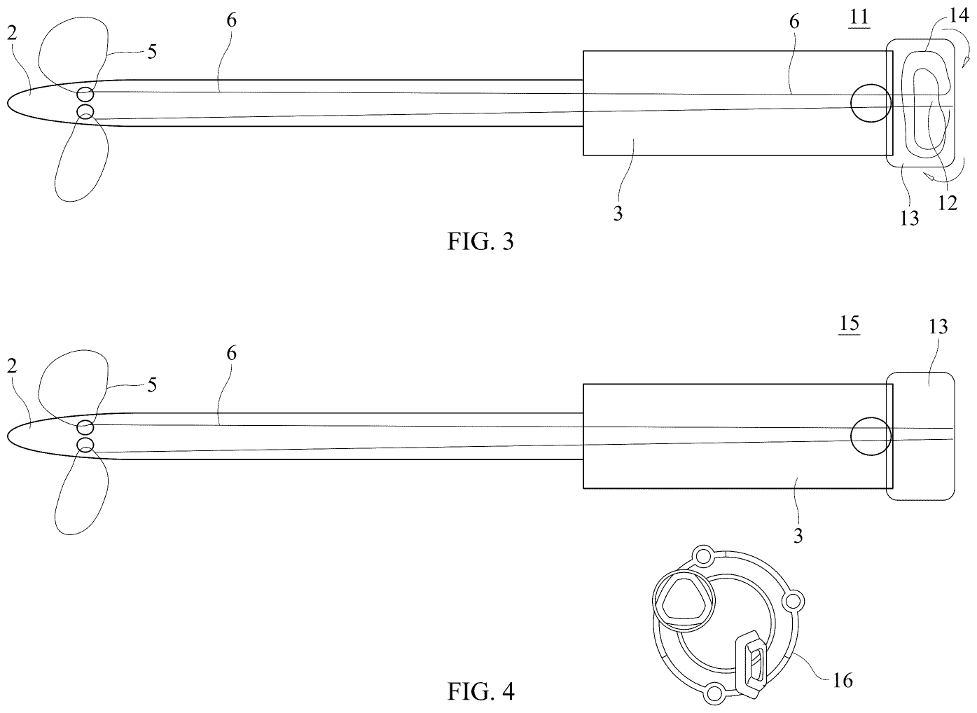

FIG. 3 depicts a third non-limiting example of an Instrument Lariat Passer device 11 in accordance with the present application, showing a different non-limiting example of a wire locking apparatus. Unless otherwise indicated, the description of the same elements from FIG. 1 or other figures or embodiments herein, will not be repeated for this and additional embodiments herein.

In FIG. 3, the wire 14 can be twisted around a rotating handle twist 12 with locking mechanism within an end portion 13 of a handle 3, as an alternative to another locking system (e.g., spoon with ratcheting teeth system in handle). As indicated above, the wire material and/or loops/lariats may be comprised of various materials, non-limiting examples of which may include wires, which may include stainless steel wire, fiber type wire, braided suture and/or FIBERWIRE®, etc.,

FIG. 4 depicts a fourth non-limiting example of an Instrument Lariat Passer device 15 in accordance with the present application, showing a different non-limiting example of a locking system or apparatus. Unless otherwise indicated, the description of the same elements from FIG. 1 or other figures or embodiments herein, will not be repeated for this and additional embodiments herein.

In particular, FIG. 4 shows examples of the present application using a BOA™ locking and ratcheting system in an end portion 13 of a handle 3 to tighten the present device and lock tight. FIG. 4 also further shows examples of a guide having a body 2, for example, as a small stainless steel metal guide that may be bullet shaped, for example at an end distal to the handle, and is configured to be capable of pushing through tissue of a patient. By way of non-limiting example, the guide body 2 may have a cross section of e.g., 1-4 mm, or 1.5-3 mm, or approximately 2 mm in diameter. In FIG. 4 (and FIGS. 1-3), the guide is magnified to show internal components.

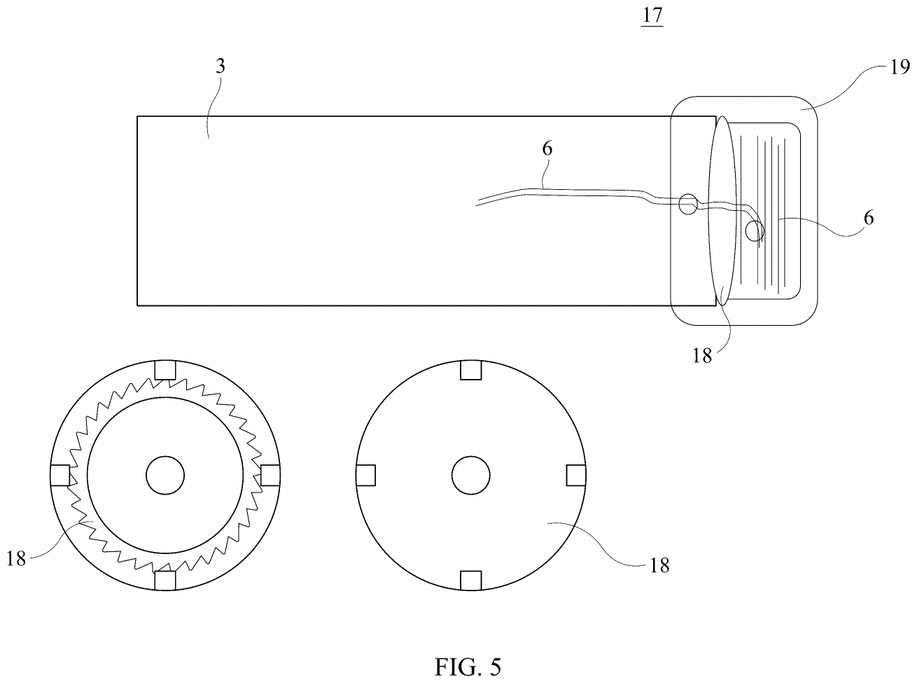

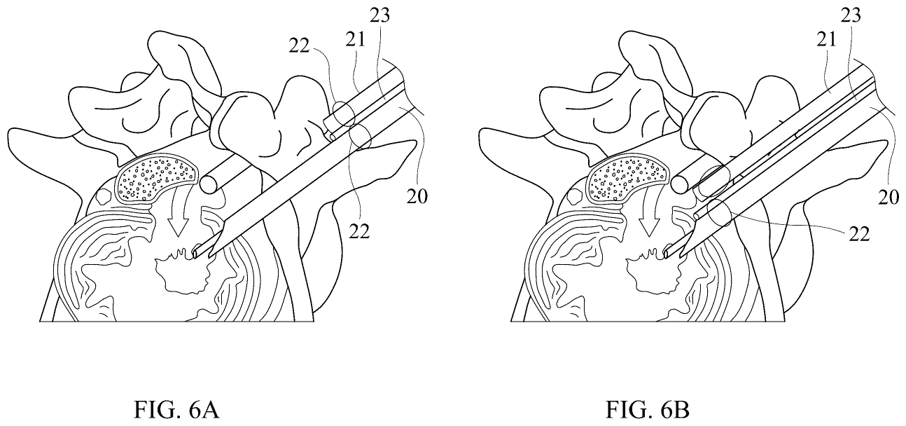

FIG. 5 depicts an optional pop-over handle 17 with ratcheting system 18 for tightening the lariat according to example embodiments of the present application. In these embodiments, a handle 19 slides over a ratcheting system 18 to lock in to tighten spool. The figure depicts the spool with ratchet system, and shows that the handle locks on allowing ratchet.

FIGS. 6A and 6B depict how the present lariat devices and systems will guide an instrument 21 to a surgical site through a small incision during minimally invasive surgery. As shown in FIG. 6A, a cannula 20 is shown that has been inserted into a patient. Above the cannula 20 is an instrument 21 to be delivered to the site. The cannula 20 and the instrument 21 both have loops 22 around them, which are part of the instrument lariat passer device 23 shown between the cannula 20 and the instrument 21. In addition to having and locking the loops 22 around the instrument 21 and the cannula 20, the Instrument Lariat Passer device 23 may push through tissue of a patient to permit the instrument 21 to be more easily inserted into a patient. The loop 22 around the instrument 21 (on top in FIGS. 6A and 6B) has more friction or is rough to stay in relatively the same position with respect to the instrument and not slide off the instrument, while the loop around the cannula may be smoother and/or looser to slide along the cannula 20 and essentially guide the instrument 21 along the cannula 20 to the surgical site as shown in FIG. 6B.

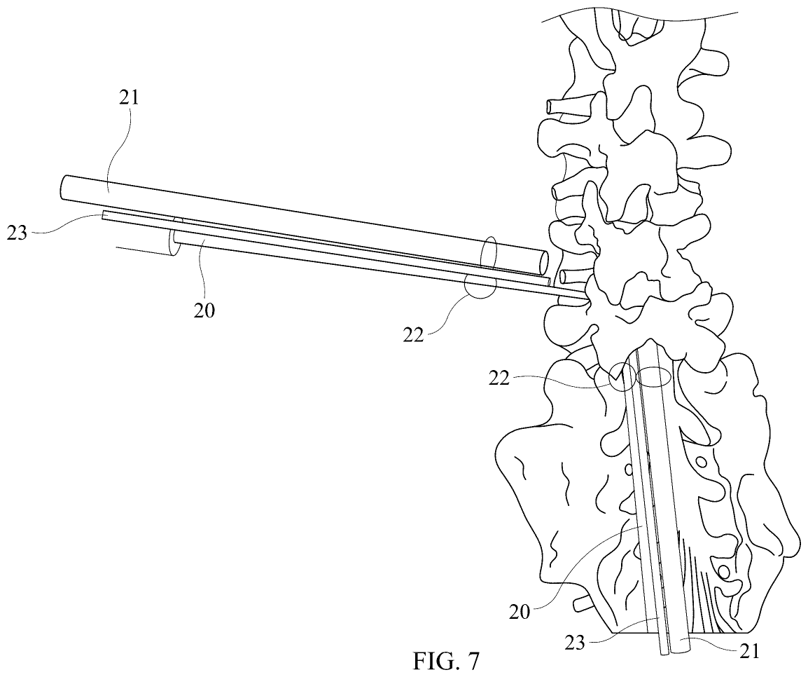

FIG. 7 depicts the insertion of an endoscope 21 with a standard medical tube or cannula 20. Again, a Lariat Passer 23 is included with the loops 22 that are part of the Lariat Passer 23 being depicted around the endoscope 21 and around the cannula 20.

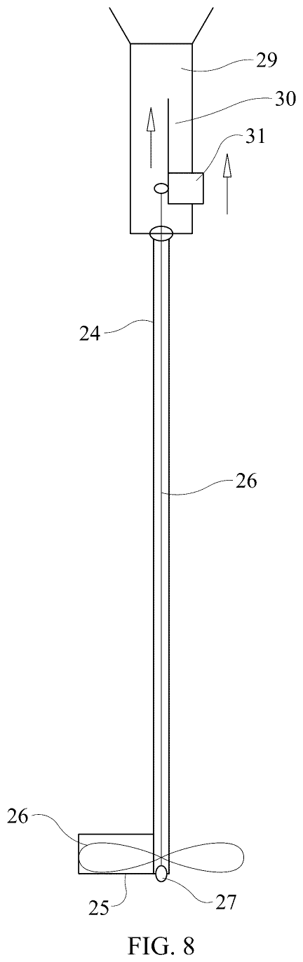

FIG. 8 shows a non-limiting embodiment of an Instrument Lariat Passer device in accordance with the present application. The depicted device comprises rigid stainless device 24, and has a fiber wire lariat 26. The depicted device also has a handle 29 and a track 30 in the handle 29. According to example embodiments, the wire slides in the track to tighten the lariat 26 around a scope and the instrument to guide the instrument to a surgical site. The present devices may include a guide 25 to guide instruments to a surgical area, through tissue. In non-limiting examples, a wire locking apparatus 31 in the handle 29 may be provided. The wire locking apparatus 31 may be reversibly lockable and unlockable, for example by sliding part of the wire locking apparatus 31 along the handle 29.

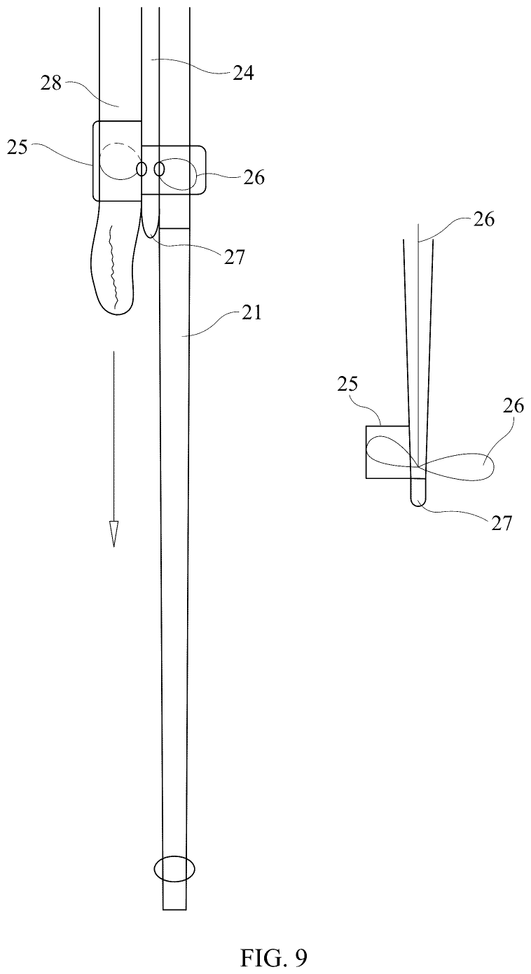

FIG. 9 depicts another example embodiment of an Instrument Lariat Passer in accordance with the present application. FIG. 9 shows a scope 21 configured to be inserted under skin of a patient, and a removable guide 25 with first and second loops 26 that go around the scope and an instrument 28, that may be used as a removable guide 25 to guide the instrument through skin of a patient to the site of the scope. In this example, the guide may be for example, a removable clip-on guide. FIG. 9 shows that the loops 26 in the Lariat Passer may be for example, FiberWire® or stainless wire to guide the instrument (though as with other embodiments, other materials are contemplated). In example embodiments, the end 27 of the removable guide 25 may have a smooth bullet shape

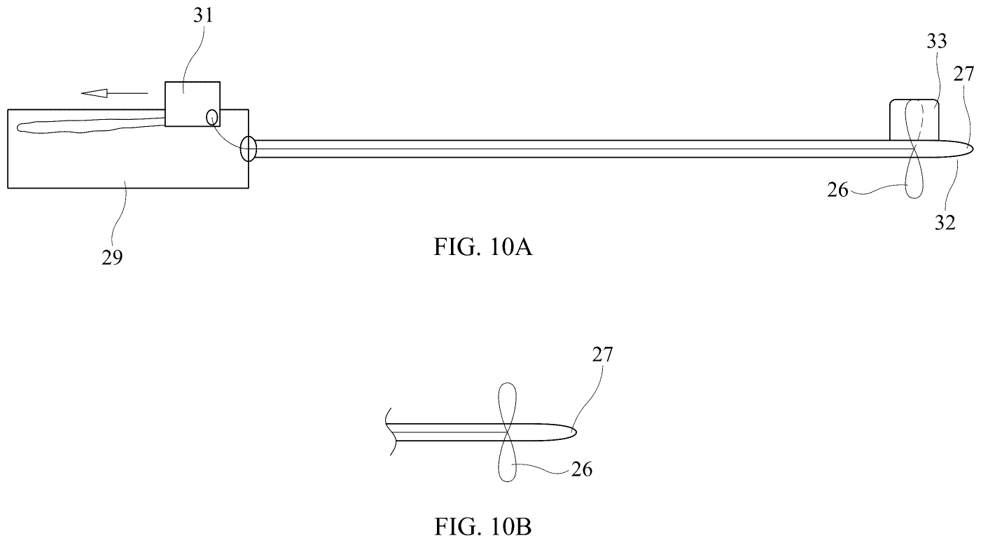

FIGS. 10A and 10B depict further example embodiments of an Instrument Lariat Passer in accordance with the present application. FIG. 10A shows a handle 29 at one end of the device opposite distal end 32. FIG. 10A shows an embodiment with a sheath 33 in which the instrument may be inserted, and FIG. 10B shows an embodiment without a sheath. Both embodiments are included herein. The sheath 33 may be used to slide the instrument in, or a fiber wire or other loop material may be used without a sheath (see e.g., FIG. 10B). FIG. 10A also depicts embodiments that may be thumb controlled. The wires (or other material) may slide in a track in the device to tighten or loosen the Instrument Lariat Passer device.

### Kits

The present application also includes kits that include one or more of the devices or systems of the present application, and optionally one or more further component(s) related to use of the present devices and systems. By way of non-limiting example, kits may optionally include one or more of the following: instructions for use, a storage container, a cannula, a visualization tool, such as an endoscopic camera, and/or one or more working instruments: such as, but not limited to, a cautery device, motorized burr, shavers, and/or biters/pituitary instruments that can be delivered accurately through the tissue.

According to example embodiments, the present devices and systems will have sterile and disposable packaging.

While the present disclosure has been described in terms of exemplary aspects, those skilled in the art will recognize that the present disclosure can be practiced with modifications that are within the spirit and scope of the present application. The examples provided herein are merely illustrative and are not meant to be an exhaustive list of all possible designs, aspects, applications or modifications of the present disclosure. Accordingly, it is intended that such changes and modifications fall within the scope of the present disclosure.

What is claimed is:

1. A lariat passer device comprising:
a guide having a body and a handle,
a first loop and a second loop at a first distal end of the guide, and
a wire extending from the first distal end of the guide to a second distal end of the guide;
wherein the handle is at the second distal end of the guide; and
wherein the handle includes a wire locking mechanism.

2. The lariat passer device of claim 1, wherein the second loop comprises a rough material or has a rough coating.

3. The lariat passer device of claim 1, wherein the first loop comprises a smoother material or has a smoother coating than the second loop.

4. The lariat passer device of claim 1, wherein the first distal end of the guide has a narrowed end.

5. The lariat passer device of claim 4, wherein the guide includes a tissue dilator.

6. The lariat passer device of claim 1, includes a wire tightening device configured to tighten the loops.

7. The lariat passer device of claim 1, further comprising a third or more loop.

8. The lariat passer device of claim 1, wherein the wire comprises at least one material selected from the group consisting of stainless steel wire, fiber type wire, and braided suture material.

9. The lariat passer device of claim 1, wherein at least one of the first loop and the second loop comprise silicon or elastic polymer.

10. The lariat passer device of claim 1, wherein the wire locking apparatus comprises a toggle spring loaded locking mechanism.

11. A kit comprising
a device according to claim 1, and
an additional component selected from the group consisting of instructions for use of the device, a storage container, a cannula, a visualization tool, and an instrument.

12. The kit of claim 11, wherein the working instrument comprises at least one instrument selected from the group consisting of a cautery device, motorized burr, a shaver, a biter, and a pituitary instrument.

13. The kit of claim 11, wherein the visualization tool comprises an endoscopic camera.

14. A method of delivering an instrument to a surgical site in a patient comprising inserting a cannula to a surgical site in a patient;
looping a first loop of a lariat passer device over the cannula;
wherein the lariat passer device comprises:
a guide having a body and a handle,
the first loop and a second loop, wherein the first loop and the second loop are at a first distal end of the guide, and
a wire extending from the first distal end of the guide to a second distal end of the guide;
wherein the handle is at the second distal end of the guide; and
wherein the handle includes a wire locking mechanism;
looping the second loop over a first instrument to be delivered to the surgical site;
tightening the first loop over the cannula and tightening the second loop around the first instrument to be delivered; and
guiding the first instrument to be delivered along the cannula to the surgical site of a patient.

15. The method of claim 14, wherein the first loop comprises a smoother material than a material of the second loop.

16. The method of claim 14, wherein the cannula comprises a camera.

17. The method of claim 14, wherein the lariat passer device further comprises a third loop, and the method further comprises looping the third loop over a second instrument to be delivered to the surgical site; tightening the third loop over the second instrument to be delivered; and guiding the second instrument to be delivered along the cannula to the surgical site of a patient.

18. The method of claim 14, wherein the method is an endoscopic surgery method.

19. The method of claim 14, wherein the surgical site is a spine of a patient.

20. The method of claim 14, wherein the surgical site is an extremity of a patient.
